(19) United States
(12) Patent Application Publication
(10) Pub. No.: US 2013/0259807 A1
(43) Pub. Date: Oct. 3, 2013

(54) CELL CULTURE SCREEN FOR AGENTS THAT CONTROL ADIPOGENESIS AND MYOFIBROBLAST DIFFERENTIATION

(71) Applicants: Mina J. Bissell, Berkeley, CA (US); Eva A. Turley, London (CA); Seyed Bahram Bahrami, Emeryville, CA (US)

(72) Inventors: Mina J. Bissell, Berkeley, CA (US); Eva A. Turley, London (CA); Seyed Bahram Bahrami, Emeryville, CA (US)

(73) Assignee: THE REGENTS OF THE UNIVERSITY OF CALIFORNIA, Oakland, CA (US)

(21) Appl. No.: 13/818,641

(22) PCT Filed: Aug. 31, 2011
(86) PCT No.: PCT/US11/50054

§ 371 Date: Jun. 11, 2013

Related U.S. Application Data
(60) Provisional application No. 61/380,125, filed on Sep. 3, 2010.
(60) Provisional application No. 61/379,265, filed on Sep. 1, 2010.

(51) Int. Cl. G01N 33/50; A61K 49/00

(52) U.S. Cl. CPC G01N 33/5073; A61K 49/0008; G01N 33/5044
USPC 424/9.2; 435/6.13; 435/34; 435/289.1; 435/7.92

(57) ABSTRACT

Methods are provided for the rapid and robust screening test agents for adipogenic activity. Agents testing positive in the assays are good candidate agents for wrinkle reduction, normalizing skin appearance after reconstructive or cosmetic surgery, e.g., grafted tissue on burn victims, normalizing skin appearance during and after wound healing, and the like. In certain embodiments the methods involve providing mammalian test cells with adipogenic potential wherein said cells are primed for, but withheld from differentiation into adipocytes; contacting the cells with the test agent(s); and screening said test cells for an adipocyte phenotype wherein the presence of a feature characteristic of an adipocyte is an indicator that said test agent is adipogenic.

### CROSS-REFERENCE TO RELATED APPLICATIONS

This application claims priority to and benefit of U.S. Ser. No. 61/380,125 filed on Sep. 3, 2010 and U.S. Ser. No. 61/379,265 filed on Sep. 1, 2010, both of which are incorporated herein by reference in their entirety for all purposes.

### STATEMENT OF GOVERNMENTAL SUPPORT

This invention was made with government support under Agreement No. LB09005060 and Contract No. DE-AC02-05CH11231 awarded by the Department of Energy. The government has certain rights in the invention.”

### FIELD OF THE INVENTION

The present invention relates to the field of pharmacological assays. In particular assays are provided that facilitate the identification of reagents that induce adipogenesis, e.g., in subcutaneous preadipocytes.

### BACKGROUND OF THE INVENTION

RHAMM (receptor for hyaluronan-mediated motility) is a hyaluronan binding protein with limited expression in normal tissues and high expression in advanced cancers. It was observed that genetic deletion of RHAMM resulted in increased subcutaneous and decreased visceral fat deposition. It was postulated that agents that block RHAMM could be used to promote subcutaneous adipogenesis and thereby selectively induce the generation of fat cells to replace those lost in the aging process. This approach could be used as a means of providing a non-surgical approach for normalizing skin appearance after reconstructive surgery, for wrinkle reduction, and for face lifts.

### SUMMARY OF THE INVENTION

Methods are provided herein to rapidly and efficiently screen test agents (e.g., putative RHAMM inhibitors) for the ability to promote adipogenesis of appropriate cells and to predict an adipogenic response in skin to those test agents.

In certain embodiments methods are provided for screening a test agent for adipogenic activity. The methods typically involve providing mammalian test cells with adipogenic potential where the cells are primed for, but withheld from differentiation into adipocytes; contacting the cells with the test agent; screening the test cells for an adipocyte phenotype where the presence of a feature characteristic of an adipocyte is an indicator that the test agent is adipogenic. In certain embodiments the cells with adipogenic potential include, but are not limited to mesenchymal stem cells, papillary and reticular dermal fibroblasts, adipose derived stem/stromal cells, preadipocytes, myeloid precursors, myogenic precursors with adipogenic potential, vascular cells, embryonic ectoderm, and embryonic mesoderm. In certain embodiments the cells with adipogenic potential are preadipocytes derived from skin, preadipocytes derived from liposuction, hair follicles, and/or preadipocytes derived from liposarcoma. In certain embodiments the cells with adipogenic potential are visceral preadipocytes (e.g., brown brown preadipocytes, white preadipocytes). In certain embodiments the visceral preadipocytes are omental or mesenteric preadipocytes. In certain embodiments the cells with adipogenic potential include subcutaneous preadipocytes. Illustrative suitable preadipocytes include, but are not limited to cells selected from the group consisting of 3T3-L1 cells, 3T3-F422A cells, 1246 cells, Ob1771 cells, TA1 cells, and 30A5 cells and/or cells derived from an animal prone to obesity or thinness. In certain embodiments the providing comprises contacting the cells with an adipocyte differentiation mix lacking at least one factor required for differentiation into an adipocyte. In various embodiments the adipocyte differentiation mix comprises one or more factors selected from the group consisting of IBMX, leptin, adponectin, glucose, adipogenic cytokine, adipogenic botanicals, dexamethasone, IGF-1, and insulin. In certain embodiments the adipocyte differentiation mix comprises one or more factors selected from the group consisting of IBMX, dexamethasone, IGF-1, and insulin. Typically, the adipocyte priming mix does not one or more of the following: insulin, IGF-1, antivirals, adipogenic cytokines, adipogenic factors, and adipogenic botanicals. In certain embodiments the adipocyte differentiation mix does not include insulin and/or IGF-1. In certain embodiments the adipocyte differentiation mix does not include an antiviral. Any of a number of screening methods are suitable. In certain embodiments the screening comprises detecting or quantifying a protein that is expressed specifically or preferentially by adipocytes (e.g., adiponectin, a lipid binding protein, and a transcription factors that promotes adipogenic transcriptomes). In certain embodiments the screening comprises detecting or quantifying lipid accumulation in the cells where accumulation of lipid indicates that the cell has acquired characteristics of an adipocyte. Lipid accumulation can be detected and/or quantified by any of a number of methods known to those of skill in the art, e.g., by detecting or quantifying lipid accumulation comprises detecting or quantifying a lipid stain. In various embodiments the screening comprises comparing the results produced by the test agent on the cells with a positive control comprising the same cell type contacted with a complete adipocyte differentiation mix (a mix that differentiates a cell having adipogenic potential into an adipocyte), where the absence of a significant difference between the test cells and the positive control is an indicator that the test agent is adipogenic. In certain embodiments the complete adipocyte differentiation mix comprises IBMX, dexamethasone, and insulin or IGF-1. In certain embodiments the screening comprises comparing the results produce by the test agent on the cells with a negative control comprising the same cell type not exposed to a differentiation mix where the absence of a significant difference between the test cells and the negative control is an indicator that the test agent is not adipogenic. In certain embodiments the test cells are disposed in a plurality of different vessels or wells in a multi-well or multi-vessel device.

The assay can take a number of formats. For example, in certain embodiments, where multiple test agents are assayed, different test agents being placed in different vessels or wells. In certain embodiments a plurality of test agents are in a single well or vessel. In certain embodiments each well or vessel containing a test agent contains a single test agent. In certain embodiments one or more vessels or wells contain positive control cells and/or one or more vessels or wells contain negative control cells. In certain embodiments the assay is carried out in a 24 well format, a 96 well format, a 384 well format, or a 1536 well format. The cell culture can be a 2-D or 3-D cell culture. Typically, the cells are grown to confluence. In certain embodiments the test cells include subcutaneous preadipocytes and visceral preadipocytes; and the screening comprises scoring as positive a test agent that induces adipogenesis in subcutaneous preadipocytes and that induces adipogenesis at a lesser amount or does not induce adipogenesis in visceral preadipocytes. In certain embodiments the assay further involves contacting fibroblasts with the test agent(s); and screening the fibroblasts for changes in myofibroblast activity, where a test agent that shows adipogenic activity and inhibition of myofibroblast activity is a candidate agent for treatment or prophylaxis of cellulite. In various embodiments the method is performed in a high throughput format.

Also provided is a cell culture system for screening a test agent for adipogenic activity. The cell culture system typically comprises one or more cell culture vessels containing mammalian cells having adipogenic potential where the cells are primed for, but withheld from differentiation into adipocytes. In certain embodiments the cells with adipogenic potential include, but are not limited to mesenchymal stem cells, papillary and reticular dermal fibroblasts, adipose derived stem/stromal cells, preadipocytes, myeloid precursors, myogenic precursors with adipogenic potential, vascular cells, embryonic ectoderm, and embryonic mesoderm. In certain embodiments the cells with adipogenic potential are preadipocytes derived from skin, preadipocytes derived from liposuction, hair follicles, and/or preadipocytes derived from liposarcoma. In certain embodiments the cells with adipogenic potential are visceral preadipocytes (e.g., brown brown preadipocytes, white preadipocytes). In certain embodiments the visceral preadipocytes are omental or mesenteric preadipocytes. In certain embodiments the cells with adipogenic potential include subcutaneous preadipocytes. Illustrative suitable preadipocytes include, but are not limited to cells selected from the group consisting of 3T3-L1 cells, 3T3-F422A cells, 1246 cells, Ob1771 cells, TA1 cells, and 30A5 cells and/or cells derived from an animal prone to obesity or thinness. In various embodiments the cells are contacted with/cultured in an adipocyte differentiation mix lacking at least one factor required for differentiation into an adipocyte. In various embodiments the adipocyte differentiation mix comprises one or more factors selected from the group consisting of IBMX, leptin, adponectin, glucose, adipogenic cytokine, adipogenic botanicals, dexamethasone, IGF-1, and insulin. In certain embodiments the adipocyte differentiation mix comprises one or more factors selected from the group consisting of IBMX, dexamethasone, IGF-1, and insulin. Typically, the adipocyte priming mix does not one or more of the following: insulin, IGF-1, antivirals, adipogenic cytokines, adipogenic factors, and adipogenic botanicals. In certain embodiments the adipocyte differentiation mix does not include insulin and/or IGF-1. In certain embodiments the adipocyte differentiation mix does not include an antiviral. In certain embodiments the cells are contacted with (cultured with) an indicator that indicates the presence of a protein that is expressed specifically or preferentially by an adipocyte (e.g., adiponectin, a lipid binding protein, and a transcription factor that promotes adipogenic transcriptomes, etc.). In certain embodiments the cells are contacted with (cultured in) an indicator that indicates the presence of lipid. In certain embodiments the cell culture system further comprises positive control cells comprising the same cell type contacted with a complete adipocyte differentiation mix. In certain embodiments the complete adipocyte differentiation mix comprises IBMX, dexamethasone, and insulin and/or IGF-1. In certain embodiments the cell culture system further comprises negative control cells comprising the same cell type not exposed to a differentiation mix. In various embodiments the test cells are disposed in a plurality of different vessels or wells in a multi-well or multi-vessel device. In certain embodiments different test agents are present in different vessels or wells. In certain embodiments a plurality of test agents are present in a single well, or each well containing a test agent contains a single test agent. In certain embodiments one or more vessels or wells contain positive control cells and/or one or more vessels or wells contain negative control cells. In certain embodiments the cell culture system comprises cells disposed in a 12 well format, a 24 well format, a 96 well format, a 384 well format, or a 1536 well format. In certain embodiments the cells are cultured in a 2-D cell culture or in a 3-D culture. In various embodiments the cells are grown to confluence. In certain embodiments the test cells comprising the cell culture system include subcutaneous preadipocytes and visceral preadipocytes. In certain embodiments cell culture system optionally includes fibroblasts. The cell culture system can be provided in a format compatible with high-throughput screening.

### DEFINITIONS

The terms “adipogenic activity” refers to the ability of an agent to induce adipogenesis, i.e., the differentiation of a cell having adipogenic potential into an adipocyte.

The term “test agent” refers to refers to an agent that is to be screened in one or more assays described herein (e.g., for adipogenic activity). The agent can be virtually any chemical compound. It can exist as a single isolated compound or can be a member of a chemical (e.g. combinatorial) library. In certain embodiments the “test agent” is not an antibody or a nucleic acid. In certain preferred embodiments, the test agent will be a small organic molecule.

The term “small organic molecule” refers to a molecule of a size comparable to those organic molecules generally used in pharmaceuticals. The term excludes biological macromolecules (e.g., proteins, nucleic acids, etc.). In certain embodiments preferred small organic molecules range in size up to about 5000 Da, more preferably up to 2000 Da, and most preferably up to about 1000 Da.

### BRIEF DESCRIPTION OF THE DRAWINGS

FIG. 1 shows that rat mesenchymal stem cells are induced to undergo adipogenesis by anti-RHAMM antibodies.

FIG. 2 shows the adipogenic effect of RHAMM peptide mimetic (15-1) and scrambled peptide control on rat mesenchymal stem cells.

FIG. 3 shows formation of subcutaneous fat pads resulting from the injection of RHAMM function blocking reagents.

FIG. 4 illustrates adipogenesis of human cells using the culture method described herein.

FIG. 5 provides a graph showing increased adipogenic effect of a test peptide and various fragments thereof.

### DETAILED DESCRIPTION

Methods are provided herein that facilitate the evaluation of one or more test agent(s) for the ability to promote adipogenesis of appropriate cells and that predict an adipogenic response in vivo (e.g., in skin or other tissues) to those test agent(s). In certain embodiments the assays identify test agents that are adipogenic in cells found in skin (e.g., subcutaneous adipocytes), but less adipogenic or not adipogenic at all in cells found in the viscera (e.g., visceral preadipocytes). Such test agents are expected to be useful for wrinkle reduction, normalizing skin appearance after reconstructive or cosmetic surgery, e.g., grafted tissue on burn victims, normalizing skin appearance during and after wound healing, while avoiding the adverse effects caused by increased visceral fat production. In cosmetic applications, unlike neurotoxin agents, which have to be injected periodically, a localized injection of adipogenic agents identified using the assays described herein, should produce long-lasting skin volumizing effects that do not involve muscle paralysis, which means there would be no loss of mobility and expression if they were to be injected into the face.

In various embodiments the screening methods typically involve providing mammalian test cells with adipogenic potential where the cells are primed for, but withheld from, differentiation into adipocytes. The cells are contacted with the test agent(s) of interest and then screened for one or more features characteristic of an adipocyte. The presence such a feature is an indicator that the test agent(s) is adipogenic. One illustrative feature characteristic of an adipocyte is accumulation of lipid which is readily detected.

It was demonstrated that a positive result in the assay is a good indicator that the test agent(s) will have similar activity in vivo (e.g., in a rat skin model, in a human, etc.) (see, e.g., Example 1). This basic assay for adipogenic activity thus identifies good candidate agents for use in normalizing skin appearance after reconstructive surgery, for use in wound healing, for wrinkle reduction, for face lifts or other cosmetic procedures, and the like.

In addition to screening for positive adipogenic activity on a test cell (e.g., a subcutaneous preadipocyte) the assays described herein can also be used to screen for the absence of such activity (or for reduced adipogenic activity on other cells). Thus, for example, a test agent can be screened for adipogenic activity on cells typically found in skin (e.g., subcutaneous preadipocytes) and on cells typically found viscerally (e.g., as visceral preadipocytes). The subcutaneous and visceral derived cells are screened for one or more characteristics of an adipogenic phenotype. Test agents that show positive activity on cells found in skin and lower activity (or no activity) on visceral cells are particularly desirable. Such test agents are expected to have beneficial effect for wrinkle reduction, normalizing skin appearance after reconstructive or cosmetic surgery, normalizing skin appearance during and after wound healing, while avoiding the adverse effects caused by increased visceral fat production. Where the test agents inhibit visceral fat production they are expected to reduce the adverse effects associated with obesity (e.g., hypertension, heart disease, obesity).

In certain embodiments the test agents can also be screened for their effect on activation of fibroblasts to differentiate into myofibroblasts. Cellulite is characterized by the deposition of fat and cellular contraction caused by myofibroblasts.

Accordingly in certain assays test agents are screened for their ability to induce adipogenesis of, for example, subcutaneous preadipocytes. The agents are also screened for activity on fibroblasts. An agent is scored as positive where it induces adipogenesis in the test cells having adipogenic potential, but has a low effect or no effect on fibroblasts, or inhibits fibroblast differentiation to myofibroblasts.

Assays for differentiation to a myofibroblast phenotype are well known to those of skill in the art and include, for example assaying cells for the expression of smooth muscle actin. Such assays include, but are not limited to, immunohistochemical assays for smooth muscle actin, reporter genes operably linked to the smooth muscle actin promoter, contractility assays, and the like.

One illustrative, but not limiting assay for smooth muscle actin in fibroblasts is described by Tanaka et al. (2001) Internat. Immunopharmacol., 1(4): 769-775. The assay was based on an enzyme immunoassay (EIA) for αSMA in microcultured fibroblasts. The αSMA produced was labeled and subjected to indirect enzyme immunoassay using alkaline phosphatase, and optical density was measured.

In various embodiments negative and/or positive control cells are included. Positive control cells are provided by exposing the same type of cells as those contacted with the test agents, to reagents (e.g., a combination of IBMX, dexamethasone, and insulin) that induce final differentiation of a cell having adipogenic potential into an adipocyte.

In various embodiments negative controls are provided culturing the same type of cells as those contacted with the test agents, in culture media that does not induce differentiation to an adipocyte.

### Cells Having Adipogenic Potential.

The cells used to evaluate the adipogenic activity of test agent(s) in the assays described herein are typically cells that have adipogenic potential. Adipogenic potential in this context refers to the ability of the cell under appropriate conditions to, substantially, or fully acquire the phenotype of an adipocyte (e.g., to substantially or fully differentiate into an adipocyte). The differentiation can be in vivo, or in vitro (e.g., upon administration of appropriate reagents).

Cells that have adipogenic potential include, but are not limited to, stem cells (embryonic stem cells, adult stem cells, induced pluripotent stem cells (IPSCs), and the like), fibroblasts, and preadipocytes. Illustrative pluripotent fibroblasts include for example, the 10T1/2, Balb/c 3T3, 1246, RCJ3.1 and CHEF/18 fibroblasts). Preadipocytes are typically unipotent (having undergone determination and being committed to an adipocyte lineage) and can remain as preadipocytes or undergo conversion/differentiation into adipocytes. Illustrative preadipocytes include, but are not limited to, 3T3-L1, 3T3-F422A, 1246, Ob1771, TA1 and 30A5 cell lines. Other suitable cell types include, but are not limited to, myeloid precursors and vascular cells with adipogenic potential.

In various embodiments the cells are characteristic of a particular region of the organism (e.g., skin, viscera, etc.) and/or are cells that characteristically differentiate into a particular fat cell (e.g., brown fat or white fat).

In certain embodiments the stem cells, fibroblasts, and preadipocytes can be derived directly from a tissue (e.g., derived from skin, derived from liposuction, derived from liposarcoma, and adipose derived stem/stromal cells) according to methods well known to those of skill in the art. For example, methods of preparing primary cultures of preadipocytes from adipose tissue are described by Crandall et al. (1999) Endocrinol., 140: 154-158, by Pask et al. (2004) Am. J. Physiol. Endocrinol. Metab., 286: E958-E962, and the like. Similarly methods of obtaining and culturing stem cells, ISPCs, and fibroblasts are well known to those of skill in the art.

Induced pluripotent stem cells (iPSCs) are obtained by re-programming somatic cells of the body. Methods of making IPSCs are well known to those of skill in the art (see, e.g., Takahashi and Yamanaka (2006) Cell, 126: 663-676; Okita et al. (2007) Nature, 448: 313-317; Wernig et al. (2007) Nature, 448: 318-324; Maherali et al. (2007) Cell Stem Cell, 1: 55-70; Nakagawa et al. (2008) Nat. Biotethnol., 26: 101-106; Takahashi et al. (2007) Cell, 131: 861-872; Yu et al. (2007) Science, 318: 1917-1920; Park et al. (2008) Nature, 451: 141-146; Huangfu et al. (2008), Nat. Biotechnol., 26(7): 795-797; Shi et al. (2008) Cell Stem Cell, 2: 525-528; Ban et al. (2011) Proc. Natl. Acad. Sci. USA, 108(34): 14234-14239; Ye. et al. (2010) Proc. Natl. Acad. Sci. USA, 107(45) 19467-19472).

In addition, stem cells, fibroblasts and preadipocytes can be obtained commercially from any of a number of suppliers. For example, visceral preadipocytes including omental preadipocytes, mesenteric preadipocytes, and perirenal preadipocytes are available from (Tebu-Bio, Ile de France, France (see, www.tebu-bio.com)). Subcutaneous preadipocytes and preadipocyte media are also available from Tebu-Bio and from ZenBio (Research Triangle Park, N.C.). Mesenchymal and dermal fibroblasts are commercially available from PromoCell Gmbh (Heidelberg, Germany). These sources of cells are intended to be illustrative and not limiting.

In various embodiments the cells (e.g. mesenchymal stem cells, skin pre-adipocytes and other cell types with adipogenic potential) are preferably low passage, maintained as subconfluent cultures and, when passaged, preferably do not exceed a dilution of 1:6.

### Priming Cells Having Adipogenic Potential.

The assays described herein involve priming “test” cells for adipogenesis, but withholding them from final differentiation into adipocytes. This can be accomplished by contacting the cells (e.g., culturing the cells in) with an adipocyte differentiation mix lacking one or more factors required to induce final differentiation into an adipocyte.

In contrast to the test cells, positive control cells are contacted with (e.g., incubated in) a complete adipocyte differentiation mix whereby differentiation into an adipocyte is induced.

Typically the test cells are exposed to the priming mix and positive controls, when utilized, are exposed to the complete differentiation mix for at least 1 hour, at least 2 hours, at least 4 hours, at least 6 hours, at least 12 hours, at least 1 day, at least 2 days, at least 3 days, at least 4 days, or longer. Generally the exposure duration is selected to be sufficient to induce differentiation of the cells if the test agent(s) have adipogenic activity.

Adipogenic Mix/Cocktail.

Confluent preadipocytes can be differentiated synchronously by a defined adipocyte differentiation mix (adipogenic cocktail). In various embodiments maximal differentiation is achieved upon treatment with the combination of insulin, a glucocorticoid (glucocorticoid agonist), an agent that elevates intracellular cAMP levels, and appropriate culture medium (e.g., medium comprising fetal bovine serum). Insulin is known to act through the insulin-like growth factor 1 (IGF-1) receptor and IGF-1 can be substituted for insulin in the adipogenic cocktail (Smith et al. (1988) J. Biol., Chem., 263: 9402-9408).

Dexamethasone (DEX), a synthetic glucocorticoid agonist, is traditionally used to stimulate the glucocorticoid receptor pathway. Other glucocorticoid agonists believed to be suitable include, but are not limited to prednisone, methylprednisone, dexamethasone acetate, dexamethasone palmitate, dexamethasone diethylaminoacetate, dexamethasone isonicotinate, dexamethasone tert-butylacetate, dexamethasone tetrahydrophthalate, and the like. Other illustrative glucocorticoid receptor agonists are described in U.S. Pat. No. 7,264,314. These glucocorticoid agonists are intended to be illustrative and not limiting. Using the teaching provided herein, one of skill in the art will recognize other suitable glucocorticoid agonists.

Methylisobutylxanthine (MIX) and 3-isobutyl-1-methylxanthine (IBMX) are cAMP-phosphodiesterase inhibitors that are traditionally used to stimulate the cAMP-dependent protein kinase pathway to increase intracellular CAMP. Other agents known to increase intracellular CAMP (e.g., U.S. Pat. No. 7,173,005) can also be used. One illustrative reagent includes serum replacement medium (e.g., KnockOut SR, Invitrogen catalog number 10828-028) plus insulin; DMEM+10% FCS and oleate (Wells et al. (2006) J. Lipid Res., 47: 450-460), and the like.

In certain embodiments adipogenic factors can also include any combination of indomethacin, PPARG gamma agonists, biotin, panthothenate, transferrin, cortisol, Tri-iodothyronine (T3), troglitazone, and/or rosiglitazone. In certain embodiments RHAMM antagonists can compensate or add to the adipogenic effects of these reagents.

The most standard adipocyte differentiation mix comprises IBMX, dexamethasone and insulin. IBMX increases intracellular cAMP, dexamethasone binds to the glucocorticoid receptor and insulin binds to the insulin receptor and/or IGF-1 receptor. These three pathways culminate in activation of the PPARγ and C/EBP family genes which activate adipocyte-specific genes encoding secreted factors, insulin receptor, and proteins involved in the synthesis and binding of fatty acids that form intracellular lipid droplets.

Priming Mix/Cocktail.

The cells having adipogenic potential can be primed but withheld from differentiation into an adipocyte by contacting them with (e.g., culturing them in) an adipocyte differentiation mix lacking one or more factors required for final differentiation into an adipocyte. In one illustrative embodiment, the priming mix/cocktail does not contain insulin (and preferably does note contain an agent that binds to the insulin receptor and/or to the IGF-1 receptor). Thus, in one illustrative embodiment, the priming cocktail comprises dexamethasone and IBMX.

In certain embodiments the priming mix can eliminate the glucocorticoid agonist (e.g., dexamethasone) or the agent(s) that stimulate intracellular CAMP.

These priming cocktails are intended to be illustrative and not limiting. Using the teachings provided herein other cocktails that prime cells for differentiation into adipocytes, but do not permit final differentiation will be available to one of skill in the art.

### Screening for Cells for One or More Characteristics of a “Differentiated” Adipocyte.

After contacting the cells with one or more test agents as described above and culturing the cells for sufficient time to permit differentiation into adipocytes, the cells are screened for an adipocyte phenotype and/or genotype where the presence of a feature characteristic of an adipocyte is an indicator that said test agent is adipogenic.

Differentiated adipocytes, also known as lipocytes and fat cells, are the cells that primarily compose adipose tissue, specialized in storing energy as fat. There are two types of adipose tissue, white adipose tissue (WAT) and brown adipose tissue (BAT), which are also known as white fat and brown fat, respectively, and comprise two types of fat cells.

Differentiated white fat cells or monovacuolar cells contain a large lipid droplet surrounded by a layer of cytoplasm. The nucleus is flattened and located on the periphery. The fat stored is in a semi-liquid state, and is composed primarily of triglycerides and cholesteryl ester. White fat cells secrete resistin, adiponectin, and leptin. Accordingly, in certain embodiments, characteristics that can be detected that are indicative of white adipocyte differentiation include, but are not limited to, lipid droplet accumulation, peripheral disposition of the cell nucleus, resistin secretion, adiponectin secretion, leptin secretion, and/or upregulation or downregulation of various other genes characteristic of differentiated adipocytes.

Brown fat cells or plurivacuolar cells are polygonal in shape. Unlike white fat cells, these cells have considerable cytoplasm, with lipid droplets scattered throughout. The nucleus is round, and, although eccentrically located, it is not in the periphery of the cell. The brown color comes from the large quantity of mitochondria. The protein expression of uncoupling protein-1 (UCP-1) is also a highly specific marker of brown adipocytes. Accordingly, in certain embodiments, characteristics that can be detected that are indicative of brown adipocyte differentiation include, but are not limited to, lipid droplet accumulation, brown cell color (mitochondrial accumulation), UCP-1 upregulation, and/or upregulation or downregulation of various other genes characteristic of differentiated adipocytes.

More generally, it is recognized that the steps of lipid droplet formation and metabolism are regulated or influenced by proteins that associate with the lipid droplets. PAT proteins (named after the founding members of the family, which are perilipin, adipose differentiation-related protein (ADFP/adipophilin/perilipin2), and TIP47 are commonly associated with lipid droplets and orchestrate their formation and maturation. PAT proteins are expressed in a tissue-specific manner, with perilipin expression restricted to adipocytes and steroidogenic cells. Again, any of these proteins can be used as a marker of adipogenesis.

Method of detecting characteristic patterns of gene regulation, expression or particular proteins characteristic of adipocytes are well known to those of skill in the art. Thus, for example, Marja-Leena et al. (1993) J. Histochem. Cytochem., 41(5): 759-764 described histochemical detection of UCP-1 protein.

Similarly, any of a variety of immunoassays can be used to detect/quantify resistin, adiponectin, leptin, or other protein markers characteristic of adipocyte differentiation. Numerous methods are also known for the detection of changes in gene expression. Such methods include, for example, in situ hybridization, real time QPCR, and the like.

Most typically, however, the easiest characteristic to detect and/or quantify is the formation of a lipid and/or a lipid droplet. When adipocytes are stained a lipophilic dye (e.g., Oil red O), the degree of staining is proportional to the amount of lipid and by implication to the extent of cell differentiation. Accordingly, in certain embodiments, the cells can be stained with a lipophilic dye (e.g., Oil red O) and the amount of dye is detected spectrophotometrically (e.g., absorbance at 510 nm). The lipophilic stain oil red O specifically stains triglycerides and cholesteryl oleate but no other lipids and provides a good measure of adipocyte differentiation (see, e.g., Ramírez-Zacarias et al. (1992) Histochem. Cell Biol., 97(6): 493-497). Other suitable markers include, but are not limited to the lipophylic dye BODIPY® (e.g., BODIPY® 12 carbon red fatty acid, Molecular Probes Invitrogen Detection Technologies Catalog No: D3822) which can be detected with fluorescent microscopy or using a fluorometer with FITC or RITC. Another common method for measuring triglycerides is a colorimetric TAG detection reagent (Thermo Electron Corp. Melbourne Australia Catalog No: 2780-400H).

It is noted that reagents and systems for the rapid quantification of lipid accumulation in cells are commercially available. For example, Vala Sciences, Inc. provides a commercial Lipid Droplet Analysis Kit containing reagents for staining and detecting lipid droplets. Vala Sciences Inc. also provides software (CYTESEER® image analysis software) for automated detection and quantitation of lipid droplets. The lipid droplet algorithm in Vala Science's CYTESEER® Image analysis platform program uses nuclear and lipid images to quantify the lipid droplets associated with each cell in the field of view.

An illustrative description of the use of this system can be found, for example, in McDonough et al. (2009) Assay Drug Dev. Technol. 7(5): 440-460 which describes the detection and quantitation of lipid droplets as well as perilipin and other markers in differentiating adipocytes.

The foregoing detection methods are intended to be illustrative and not limiting. Using the teachings provided herein, other methods of detecting adipocyte differentiation will be available to one of skill in the art.

### Test Agents

The agent can be virtually any chemical compound. It can exist as a single isolated compound or can be a member of a chemical (e.g. combinatorial) library. Illustrative test agents include, but are not limited to proteins, peptide mimetics, nucleic acids (e.g., siRNA), lectins, antibodies, small organic molecules, and the like.

In certain embodiments the test agents include compounds believed to be or suspected of having adipogenic activity. Such compounds include, but are not limited to for example, upregulators of CAMP, glucocorticoid analogues, peroxisome proliferator-activated receptor (PPAR) binders, and the like.

In certain embodiments the test agent include compounds believe to be inhibitors of RHAMM expression and/or activity. Such agents include, but are not limited to anti-RHAMM antibodies, RHAMM binding lectins, RHAMM siRNA, RHAMM intrabodies, RAHMM ribozymes, RHAMM synthetic peptides, RHAMM mimetic peptides, hyaluronan mimetic peptides, and the like.

In certain embodiments the test agents include molecules with no a priori known or suspected activity.

### Assay Formats.

The assays described herein can be performed in any of a number of formats. In certain embodiments the cells are cultured in multi-well plates (e.g., a 12 well format, a 24 well format, a 96 well format, a 384 well format, a 1536 well format, etc.) as a 2-dimensional (2D) cell culture. Two-dimensional culture systems are simpler (than 3D systems), require less manual intervention, and are well suited to high-throughput screening (HTS) systems.

In various embodiments one or a multiplicity of different test agents are assayed at the same time. In various embodiments single test agent is placed in each well. Optionally, additional wells can function as positive controls (cells treated with an adipocyte differentiation mix). Optionally additional wells can function as negative controls (e.g., cells not contacted with a differentiation mix). In addition, multiple cell types (e.g., visceral adipocytes, subcutaneous adipocytes, etc.) can be screened simultaneously with different cell types in different wells.

In addition, different wells can provide different assays. Thus, for example, certain wells can be used to assay lipid accumulation, while other wells are used to assay for perlipin or actin production. It is also possible to obtain multiple readouts from a single well. Accordingly, it is possible to assay lipid accumulation, perlipin colocalization, actin expression and other parameters in a single well using for example, different indicator reagents.

To facilitate screening of large numbers of test agents, in certain embodiments, multiplexed assays are performed. In such assays, multiple test agents are places in each well. The agents(s) used in wells that show a positive result are then tested individually or in subcombinations to determine which of the multiple test agents produced the desired effect.

A particular assay format may be determined, for example, by the cell types to be assayed, the readouts desired, and the number of test agents.

These assay formats are intended to be illustrative and not limiting. Using the teaching provided herein numerous assay formats will be available to one of skill in the art.

### Scoring.

In various embodiments, the assays described herein are deemed to show a positive result, when exposure to the test agent(s) results in one or more characteristics of an adipocyte phenotype in the test cells. In certain embodiments the assays are deemed to show a positive result when exposure to the test agent(s) results in one or more characteristics of an adipocyte phenotype (e.g., lipid accumulation) in test cells characteristic of skin (e.g., subcutaneous preadipocytes) and a lower effect or no effect in test cells derived from visceral tissue (e.g., visceral preadipocytes).

In certain embodiments this is determined with respect to the level measured or known for a positive control (e.g., cells exposed to a complete adipogenic mix/cocktail) and/or a negative control (e.g., cells not exposed to an adipogenic or priming mix/cocktail). In one embodiment, the assay is deemed to show a positive result (e.g., adipogenic activity of a test agent) when the difference between sample and negative “control” is statistically significant (e.g. at the 85% or greater, preferably at the 90% or greater, more preferably at the 95% or greater and most preferably at the 98% or 99% or greater confidence level) and/or when the difference between sample and positive control is not statistically significant.

### High-Throughput Screening.

The assays described herein are amenable to “high-throughput” modalities. Conventionally, new chemical entities with useful properties (e.g., adipogenic activity on subcutaneous preadipocytes) are generated by identifying a chemical compound (called a “lead compound”) with the desirable property or activity (e.g., inhibition of RHAMM expression and/or activity), creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one embodiment, high throughput screening methods involve providing a library containing a large number of compounds (candidate compounds) potentially having the desired (adipogenic) activity. Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

Combinatorial Chemical Libraries

In certain embodiments, combinatorial chemical libraries can be used to assist in the generation of new chemical compound leads. A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) 37(9): 1233-1250).

Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the assays described herein. Other chemistries for generating chemical diversity libraries can also be used. Such chemistries include, but are not limited to: peptoids (PCT Publication No WO 91/19735), encoded peptides (PCT Publication WO 93/20242), random bio-ligomers (PCT Publication WO 92/00091), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a Beta-D-Glucose scaffolding (Hirschmann et al., (1992) J. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries (see, e.g., Strategene, Corp.), peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), PPAR inhibitor libraries (see, e.g., Eanamine, Ltd.), and small organic molecule libraries (see, e.g., benzodiazepines, Baum (1993) C&EN, January 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the methods described herein. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

High Throughput Assays of Chemical Libraries.

The assays for adipogenic activity described herein are amenable to high throughput screening. Certain preferred assays detect increases differentiation of adipocytes by detection of lipid accumulation and/or by the upregulation of characteristic protein markers resulting from contact with the test agent(s).

High content analysis (HCA) is a technology in which candidate pharmaceuticals or genomic (e.g., RNAi or cDNA) libraries, or antibody libraries, or peptide libraries, etc., are tested for potential beneficial effects via assays performed on cells cultured on microtiter plates (see, e.g., Aza-Blanc et al. (2003) Mol Cell. 12(3): 627-637; Berno et al. (2006) Meth. Enzymol. 414: 188-210; Bettencourt-Dias et al. (2004) Nature, 432: 980-987; Carpenter and Sabatini (2004) Nat. Rev. Genet. 5(1): 11-22; Cho et al. (2006) Cell Metab. 3(5): 367-378; Harada et al. (2005) Genome Res., 15(8): 1136-1144; Huang et al. (2004) Proc. Natl. Acad. Sci., USA, 101(10): 3456-3461; Iourgenko et al. (2003) Proc. Natl. Acad. Sci., USA, 100(21): 12147-12152; Marcelli et al. (2006) J. Cell Biochem., 98(4): 770-788; Mukherji et al. (2006) Proc. Natl. Acad. Sci., USA, 103(40): 14819-14824; Rines et al. (2006) Meth. Enzymol. 414: 530-565; Sharp et al. (2006) J. Cell Sci. 119: 4101-4116; Zheng et al. (2004) Proc. Natl. Acad. Sci., USA, 101(1): 135-140L Dragunow (2008) Nat. Rev. Neurosci. 9(10): 779-788). The cells can then be stained or labeled to visualize structures or proteins and photographed via robotic digital microscopy workstations.

The images are analyzed for information by algorithms designed to identify and extract information relevant to a particular cell/disease model (see, e.g., 66. Haney et al. (2006) Drug Discov. Today, 11: 889-894; Giuliano et al. (2006) Meth. Enzymol. 414: 601-619; Nicholson et al. (2007) ACS Chem Biol. 2(1): 24-30; and the like). Advances in automatic acquisition, measurement, comparison, and pattern classification facilitate the detection and/or quantitation of numerous cellular parameters including, but not limited to morphological parameters, protein levels, gene expression, and the like. Digital images from conventional and confocal microscopy can be analyzed by sophisticated image-analysis algorithms permitting quantitative approaches to microscopy-based cellular phenotypic characterization (see, e.g., Tarnok (2006) Cytometry A. 69(7): 555-562; Carpenter (2007) Nat Meth. 4(2): 120-121). Thousands of images representing hundreds of thousands of individual cells can be acquired via HCA workstations in a single experimental session permitting the rapid screening of hundreds of thousands of compounds.

Numerous vendors offer microscope-based instruments capable of producing images of fluorescent labeled components of cells grown in microtitre plates. These instruments are typically bundled with analysis software capable of defining the relative distribution of several fluorescent markers on a cell by cell basis. As the readers have improved and image acquisition and analysis times have reduced, the potential for screening larger compound libraries has presented itself. High Content Screening (HCS) i.e. the generation of multi-parameter data from a single well has thus become an important tool in the High-Throughput Screening (HTS) laboratory.

HCA analysis for particular markers of adipocyte differentiation is known to those of skill in the art. For example, McDonough et al. (2009) Assay Drug Dev. Technol. 7(5): 440-460, described HCA screening for adipocyte differentiation using Vala Sciences, Inc. commercial Lipid Droplet Analysis Kit containing reagents for staining and detecting lipid droplets and CYTESEER® image analysis software for automated detection and calculation of lipid droplets. In one experiment described therein the same field of view was imaged in three separate optical channels, to selectively visualize the nuclei, lipid droplets, and protein. Lipid droplets were quantitated using the CYBERSEER® software. In addition, colocalization of a protein (perilipin) was also determined.

A large number of high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

These high-throughput systems and examples are intended to be illustrative and not limiting. Using the teachings provided herein, the assays described herein can readily be implemented on numerous other HTS/HCA analysis systems.

### Cell Culture Systems.

In various embodiments cell culture systems are provided for performing the assays described herein. In certain embodiments the cell culture systems comprise one or more cell culture vessels containing mammalian cells having adipogenic potential where the cells are primed for, but withheld from differentiation into adipocytes. In various embodiments the cells are in acute culture while in other embodiments, the cells are established cell lines that have been passaged numerous times. Illustrative cells include, but are not limited to mesenchymal stem cells, papillary and reticular dermal fibroblasts, adipose derived stem/stromal cells, preadipocytes, myeloid precursors, vascular adipocyte precursors, and the like. In various embodiments the cells are provided in an adipocyte differentiation mix lacking at least one factor required for differentiation into an adipocyte.

The cell culture systems can be provided in a number of formats. For example, In certain embodiments the systems are provided in a multi-well or multi-vessel device (e.g., in a 12 well format, a 24 well format, a 96 well format, a 384 well format, or a 1536 well format and the like). In certain embodiments the culture system is provided in a format compatible with a particular HTS and/or HCA system.

### Animal Models for Screening.

In certain embodiments test agent(s) that show a positive result in the cell-based (in vitro) assays described above, are further validated in an in vivo animal model. For example, the fidelity of the screen for identifying reagents that are effective promoters of adipogenesis in vivo can be tested by screening the test agent(s) for their ability to promote subcutaneous fat accumulation in an animal model (e.g., when injected under the skin of 7-month old female rats). In certain embodiments injection in the outer ear is performed. It has been observed that injection at this site provided good data. It has been found that the ability of test agent(s) to promote mesenchymal stem cell differentiation into adipocytes matches very closely the ability of these reagents to promote subcutaneous fat accumulation in rat skin (or other animal models) in vivo.

### Kits.

In certain embodiments kits are provided for practice of the assays described herein. In certain embodiments the kits contain one or more cell types having adipogenic potential (e.g., preadipocytes). The kits can additionally include a reagent mix to prime the cells for adipogenesis, but withheld them from final differentiation into adipocytes. The kits can additionally contain media for propagating and/or maintaining the cells. The kits can additionally include one or more reagents for detecting differentiated adipocytes and/or software for facilitating such detection. The kits can optionally include any reagents and/or apparatus to facilitate practice of the assays described herein. Such reagents include, but are not limited to buffers, labels, labeled antibodies, labeled nucleic acids, filter sets for visualization of fluorescent labels, blotting membranes, and the like.

In addition, the kits can optionally include instructional materials containing directions (i.e., protocols) for the practice of the assay methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Adipogenesis Assay

### Cell Types:

In certain embodiments any pre-adipogenic cell, dermal fibroblasts, pluripotent fibroblast (e.g. 3T3-L cell), or mesenchymal stem cell is suitable for this assay.

### Materials

Cells can be cultured in a 96 well or other multiwell tissue culture plate.

Adipogenesis Initiation/Priming Medium:

To prepare adipogenesis initiation/priming medium, a IBMX standard solution (0.5 mM) is diluted 1:1000 and a dexamethasone standard solution (1 μM dexamethasone standard solution (in DMSO)) is diluted 1:10,000 in DMEM+10% FCS. A typical cocktail of antibiotics can be added to restrict microbe growth. This initiation medium can be stored for up to 6 weeks at 4° C.

Adipogenesis Progression Media:

To prepare positive control progression medium, a standard insulin solution (10 μg/ml insulin) is diluted 1:1000 in DMEM+10% FCS. Antibiotics of choice are added to control growth of microbes. This control progression medium can be stored for up to 6 weeks at 4° C.

To prepare experimental/test (e.g., RHAMM blocking or other experimental adipogenic promoting reagent) progression medium, test agents are provided in a standard solution (e.g. 0.001-10 μg RHAMM antibody, peptide mimetic(s), or other test agent(s)). The test agent solution is diluted to the desired concentration (e.g. 1 ng-10 μg RHAMM peptide) in DMEM+10% FCS.

Adipose maintenance media can include the following: DMEM+10% FCS as a positive control, DMEM+10% calf serum (CS) as a negative control, and DMEM+10% FCS+adipogenic reagents (e.g. RHAMM antibody, peptide, other test agent(s), etc.) as the experimental/test medium.

### 3D Culture Assay

One suitable 3D adipogenesis assay is a modification of the assay described by Keck et al. (2011) Burns, 37: 626-630). Pre-adipocytes including human skin pre-adipocytes, reticular fibroblasts, mesenchymal stem cells and other cell types with adipogenic potential are cultured until confluence on tissue culture plastic surfaces in DMEM (low glucose)+10% fetal bovine serum supplements. Cells are exposed to a cocktail of factors that promote adipogenesis until they differentiate into adipocytes. The differentiated adipocytes are maintained in DMEM (low glucose)+10% FCS and matrigel is layered on top of the adipocyte monolayers. Reticular and papillary fibroblasts are seeded onto the surface of the polymerized matrigel layer at low density (e.g. 5×10³ cells/ml) and allowed to invade into the matrigel. Keratinocytes (primary or cell lines) are seeded on the surface of the matrigel and then supernatant culture medium is removed so that keratinocytes spread onto the surface of the matrigel layer and keratinize. This assay results in the formation of three layers typical of skin: differentiated subcutaneous fat layer, dermal layer containing fibroblasts and a surface layer of differentiated keratinocytes. This assay is particularly useful for assessing the factors that affect differentiation of human pre-adipocytes under conditions that resemble or model those of intact skin.

Injection of Peptides into Rats and Nude Guinea Pigs.

The effect of RHAMM function blocking antibody (R-6836-B) and RHAMM synthetic Peptide B on subcutaneous adipogenesis in Nude guinea pigs was evaluated using the following reagents: 1) Anti-RHAMM antibody (R-6836-B) 0.025 mg/ml); 2) RHAMM synthetic peptide (1 mg/ml); 3) Rat tail Type I collagen (1 mg/ml); and 4) Sodium bicarbonate. In certain embodiments the amount of RHAMM peptide and antibody range from about 0.1 to about 250 μg/ml, more preferably from about 0.25 to about 100 μg/ml.

Sample Preparation:

The pH of the collagen solution was adjusted to 7-8 by adding sodium bicarbonate solution (add 30 μl of sodium bicarbonate to 1 ml of collagen solution). During the process the collagen solution was kept on ice.

Anti-RHAMM antibody was added to collagen solution to prepare different concentrations (0.25 and 2.5 μg of antibody/ml solution). Also RHAMM synthetic Peptide B was added to collagen to prepare 10 μg and 100 μg of peptide B/ml solution.

Injection Process:

One animal for each condition was used (total of four nude guinea pigs and two sprague dawley rats). Five locations of each nude guinea pigs was injected with reagent (two in the back, two in the stomach near to mammary fat pad, and one in back of the neck). A 1 ml syringe with G20-G22 hypodermic needles was used for injection. Animals were sedated with isoflurane gas (level 1-1.5 mixed with O₂). The injection site of animal was cleaned with an alcohol wipe (the injection sites on the sprague dawley rat were shaved prior to injection). 1 ml of collagen solution containing 0.25 μg of antibody was injected very slowly under the skin in back or stomach of the animal. After each injection each animal was remained in the same position for up to 5 minutes. The injection site was marked with permanent sharpie pen.

The same procedure was performed with 2.5 μg/ml of antibody, as well as 10 μg/ml and 100 μg/ml of RHAMM peptide B.

For controls, (for each injection) 1 ml of collagen only (without RHAMM reagent) was injected in the contralateral sites.

Two rats were used as positive controls: One was injected with known optimal dose of RHAMM antibody (2.5 μg/ml), and the second with the known optimal dose of RHAMM synthetic peptide B (100 μg/ml). Animals were housed as usual for 7 days.

### Culture Screening Assay

Maintenance of Cells

Maintain cell stocks as per instructions from cell line provider. For best results maintain cells in DMEM+10% CS rather than FCS. Routinely keep cells at low culture density (e.g. 4×10⁵ cells/100 mm dish) to reduce background adipogenesis that occurs with culture confluence.

Multiwell Assay

Cells for the adipogenesis screen can be plated as follows: The cells are trypsinized and then resuspended in 1 ml DMEM+10% CS to neutralize the trypsin. The cells are then counted. In certain embodiments approximately 30,000 cells/ml DMEM+10% CS are plated. The cells are left in this medium for 1-2 days. Certain wells, e.g., a row of wells without cells can be kept to provide blank(s).

The DMEM+10% CS is removed and replaced with appropriate amount (for the size of multiwell dishes) of initiation medium. For negative controls, a negative control medium (e.g., DMEM+10% calf serum (CS) described above as a negative control) can be used. The cells are incubated for 48 hours at 37° C., 5% CO₂.

The adipogenesis assay is removed from culture and positive control or experimental reagent progressing medium is added. The cells are incubated as above for 48 hours.

The progressing medium is then removed and replaced with maintenance medium. Negative controls should be maintained in negative control maintenance medium.

The cells can then be left for, e.g., 5-7 days then either BODIPY® dye (25 μM) or 1% Oil Red O is added for 15 minutes. The cells are then gently washed in phosphate buffered saline.

The dye can be extracted with methanol:ethanol (1:1) mixture and read oil red O at 520 nm using an ELISA plate reader or for BODIPY® using a fluorometer that detects FITC.

This method can be adapted to co-stain for other molecules such as smooth muscle actin (detected by labeled anti-smooth muscle actin antibody). The staining for a second or third molecule would be performed concomitant with lipid or can be conducted on extracted cells. If the latter method is used, after lipid extraction, cells should be fixed in 3% freshly prepared paraformaldehyde in PBS. Staining for the additional molecules is then conducted according to the methods of the antibody manufacturer.

In various embodiments of the adipogenesis assays monolayers of cells with adipogenic potential are covered with a layer of agarose (0.3%-1%, low melting temperature agarose dissolved in culture medium, e.g. SeaPlaque Agarose, Lonza) that contains the adipogenic cocktail. Culture medium (e.g. Dulbecco's Modified Eagle's Medium (DMEM) or DMEM/Ham's medium mixture) supplemented with 15% fetal bovine serum supplements is layered on top of the polymerized agarose. This method promotes adipogenesis because the agarose permits sustained slow release of adipogenic factors and prevents the rolling up of cell monolayers, which would hinder use of assay for high throughput analysis.

In addition, pre-adipocytes can be placed on glass coverslips or adhesive proteins such as fibronectin to reduce detachment of monolayers (rolling up) and promote adipogenesis.

### Results.

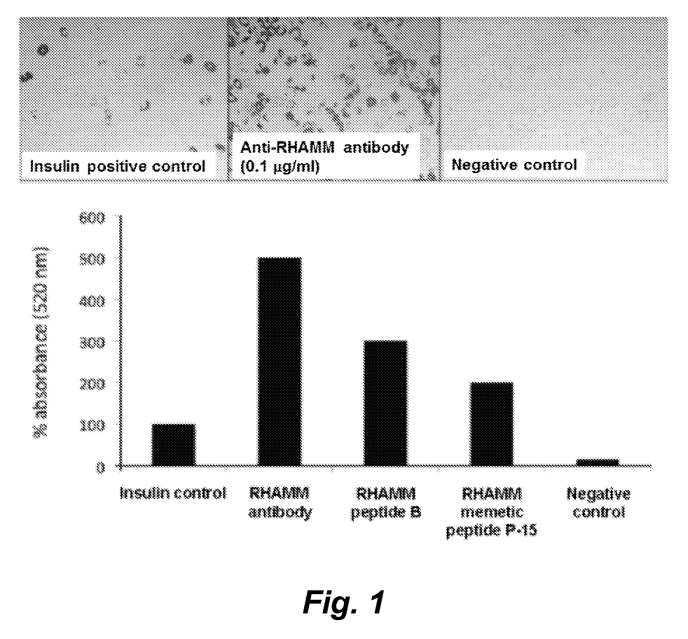

Rat mesenchymal cells and mouse embryonic pre-adipogenic fibroblasts (e.g. 3T3-L cells) underwent adipogenesis when exposed to insulin in the progressing medium (rat mesenchymal stem cells shown in FIG. 1). The extent of lipid accumulation was quantified by measuring the amount of Oil Red O, detected at 520 nm and is shown as 100%. The effect of a RHAMM function blocking antibody (anti-peptide B antibody) on adipogenesis is shown in FIG. 1, top panel, and quantified, together with effects of RHAMM peptide B and RHAMM mimetic peptide P15 in an ELISA (results shown in FIG. 1, bottom panel). The effect of the RHAMM anti-peptide B antibody is 5 times greater than insulin while RHAMM peptide sequence B and mimetic peptide are 3 ns 2 times greater respectively. Cells in the negative control were grown in DMEM+CS and were not exposed to either initiating or progressing medium.

In an earlier screen tested a combination of 20 peptides and antibodies to RHAMM. Three test agents (anti-peptide B, peptide B and peptide P-1) in this screen were adipogenic and the extent to which they were adipogenic in the screen was replicated in 3D and in vivo. In addition, rat and human cells were equally good in predicting adipogenesis in vivo.

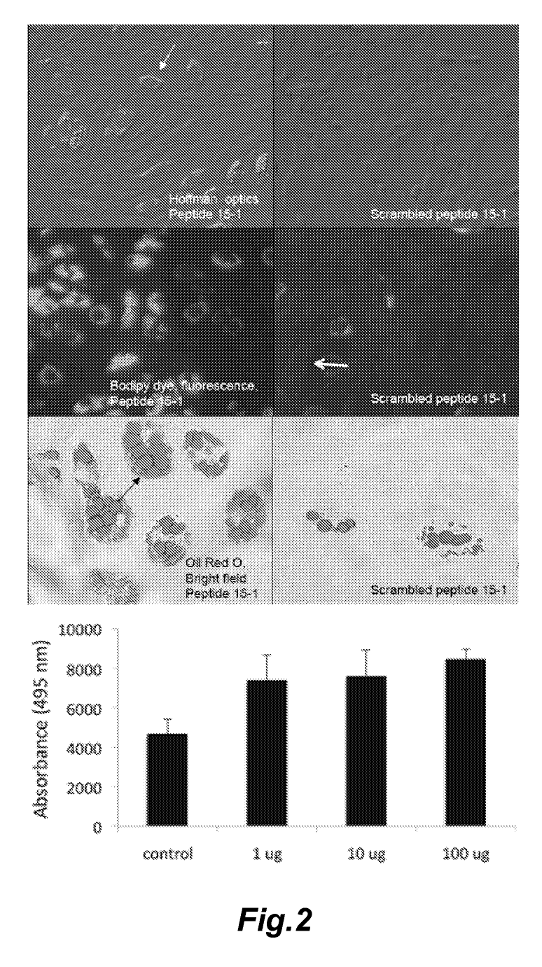

FIG. 2 shows the effect of RHAMM peptide mimetic P15-1 on adipogenesis of rat mesenchymal stem cells using a control for the RHAMM reagent, which in this case is a scrambled peptide sequence of 15-1. Hoffman optics reveals the presence of the cell monolayer. Lipid droplets can be seen in a number of cells and lipid droplets are shown by bodipy uptake (green fluorescent dye, arrow) and oil red O uptake (red droplets, arrow).

The graph shows a dose response curve for the P-1 peptide, isolated using an unbiased screen, B-1 peptide rationally designed based on known molecular interactions between RHAMM and its ligands, and a RHAMM antibody were assayed for their effects on pre-adipocyte stem cells and fibroblasts in culture and when injected into the dermis of aged rats. Reagents were ranked on a scale of 0-5, with 5 representing the highest possible score.

TABLE 1
The adipogenic potential of reagents in culture and rat assays.

| | Adipogenic Response | | |
|---|---|---|---|
| | 2D Rat | In vivo | 2D Human |
| Reagent | Preadipocytes | Rat Skin | Preadipocytes |
| P-1 peptide | 2 | 2 | 2 |
| B-1 peptide | 3-4 | 4 | 3 |
| Antibody | 5 | 5 | 4 |

Adipogenic Effect of RHAMM Mimetic Peptide 15-1 Using Uptake of BODIPY® Dye to Detect Lipid.

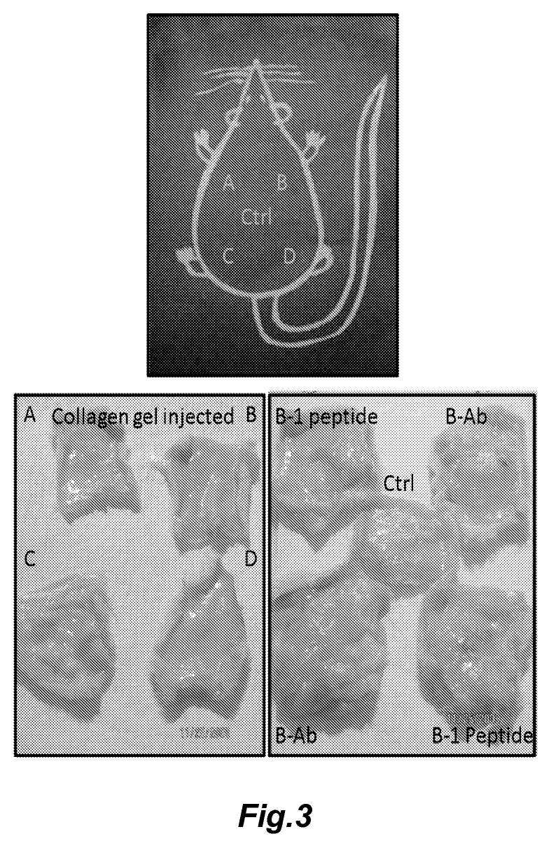

The fidelity of the screen for identifying reagents that are effective promoters of adipogenesis in vivo was tested by comparing the ranking of reagents in the culture screen with their relative ability to promote subcutaneous fat accumulation when injected under the skin of 7-month old female rats. Subcutaneous fat pad accumulation resulting from injection of RHAMM function blocking reagents in rats is shown in FIG. 3. The ability of reagents to promote mesenchymal stem cell differentiation into adipocytes matches very closely the ability of these reagents to promote subcutaneous fat accumulation in rat skin in vivo.

Using the culture screening method, we showed that several human skin pre-adipocyte cell lines and reticular dermal fibroblasts have adipogenic control. Generic human fibroblasts (from foreskin) and skin papillary fibroblasts did not exhibit adipogenic potential in this assay. Thus the screening method can also be used to selectively identify adipocyte stem cell populations.

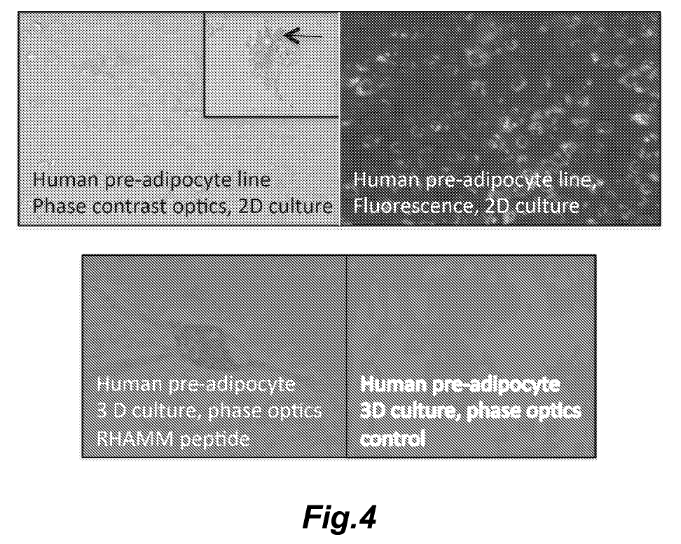

RHAMM reagents identified by the culture screen described above were also tested for their ability to promote adipogenesis in human skin cells, grown in the 2D method of the culture screen and also grown in 3D (FIG. 4). Results in FIG. 4 show that RHAMM peptide B promotes adipogenesis in a human pre-adipocyte cell line, when grown using culture screen methods or when grown in a 3 dimensional culture environment using collagen type I gels. RHAMM reagents identified as being pro-adipogenic by the culture screen have been tested in a nude guinea pig model. This model is becoming increasingly used to model human skin since it resembles human facial skin in that there is no hair, the keratinocyte layer is multilayered (unlike the very epidermal layer of rats and mice) and the subcutaneous fat layer is very thin. In this model, RHAMM agents identified as being proadipogenic in the assays described herein increased subcutaneous fat in the animal model.

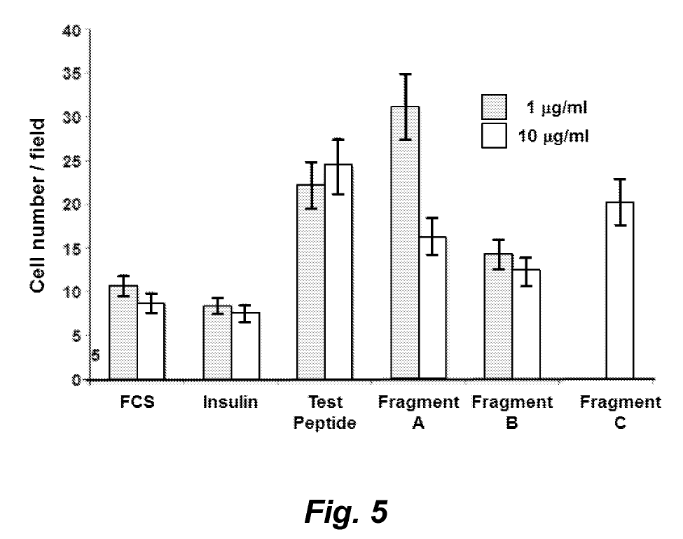

The results presented in the graph in FIG. 5 show an increased adipogenic effect of a test peptide when rat mesenchymal stem cells are induced by indomethacin (100 μM), insulin (10 μg/ml), dexamethasone (1 μM), and IBMX (0.5 mM). This graph also show that certain fragments of the peptide are as active or more active than the entire test peptide.

### Example 2

### 2D Adipogenesis Differentiation of Pre-Adipocyte

The commercial kit fro GIBCO (STEMPRO® Adipogenesis Differentiation Kit) to differentiate preadipocyte or adipose derived stem cells or mesenchymal stem cells (MSCs).

### Adipogenesis Differentiation

Low passage adipose derived stem cells and MSCs (<8 to 10 passages) offer stronger multipotency. Passaging should take place when cultures reach 60 to 80% confluency (confluency also can reduce multipotency).

The cells are cultured in standard growth medium and ensured of mid-log growth phase confluence (60 to 80%). Suitable growth medium is DMEM:Ham's F-12 (1:1) supplemented with 10% FCS (MSC Qualified), 200 mM L-glutamine, and 10 mg/ml Gentamicin).

The medium and floating cells are then aspirated and from the culture flask and discarded. 5 to 10 mL DPBS is added and the cell monolayer is gently rinsed.

The DPBS is removed and 1-2 mL of pre-warmed trypsin (%0.25) is added. The cells are incubated for 2 minutes at 37° C. or until the cells have fully detached. Then 4 ml growth media is added to neutralize the trypsin. The detached cells can be gently pipetted into a single cell solution.

The cell suspension is removed from the container (e.g., flask) and transferred into a centrifuge tube. The cells are pelleted at 100×g for 5 to 10 minutes.

Cell viability and total cell density can be determined using, e.g. trypan blue stain and a manual or automated hemocytometer cell counting method.

The pellet is then resuspended in an appropriate volume of pre-warmed growth medium. The cells are then seeded at e.g., 1×10⁴ cells/cm² and incubated in growth media at 37° C. 5% CO₂ for a minimum of 2 hours up to 4 days.

The media is then replaced with pre-warmed adipogenesis differentiation medium and incubation is continued. Cells will continue to undergo limited expansion as they differentiate under adipogenic conditions. The cultures can be re-fed every 3 to 4 days.

For adipogenesis differentiation medium, commercially available media from Gibco or ZenBio can be used or an adipogenic cocktail can be formulated as follows: DMEM:Ham's F-12 (1:1) supplemented with, 3% FCS, 200 mM L-glutamine, 10 mg/ml Gentamicin), (100 nM) insulin, 0.2 nM T3, 1 μM dexamethasone 0.25 mM IBMX and 1 μM rosiglitazone.

After 7 to 14 days adipogenic cultures can be processed for gene analysis or staining with Oil Red O or, e.g., BODIPY®.

### Example 3

### 3D Culture and Adipose Differentiation of Pre-Adipocyte

Prepare pre-mix solution according to Table 2 below, preferably the day before use and no more than one week in advance. Prepare this solution on ice and filter sterilize with a 0.22 μm filter before use and adjust the pH=7.5 with 1N HCl solution.

TABLE 2
Premix solution.

| | | |
|---|---|---|
| Pre-mix solution | 1 well | 6 wells |

| | | | | |
|---|---|---|---|---|
| 5x DMEM:5x Ham's F-12 (1:1) | 395 | μl | 2,400 | μl |
| L-Glutamine | 39 | μl | 234 | μl |
| Gentamicin | 5 | ml | 30 | μl |
| NaHCO₃ | 120 | μl | 730 | μl |
| FCS | 440 | μl | 2,600 | μl |
| Total volume: | 1 | ml | 6 | ml |

Culturing Pre-Adipocyte in 3D Collagen System

All the reagents are pre-cooled. Human preadipocyte cells are harvested with <80% confluency and the cell number is determined. Adipogenesis differentiation medium is then added to the cells and the cell concentration is adjusted for seeding density of e.g., 2×10⁵ cells per ml media.

In a 50-ml falcon tube, bovine tail collagen (1.1 mg/ml) is combined with premix solution and swirled on ice to mix well. Cells in adipose differentiation medium are added into the collagen mixture, swirling gently to avoid air bubbles.

For adipogenesis differentiation medium the commercial brand from GIBCO or ZenBio can be used or differentiation media with the following components: DMEM:Ham's F-12 (1:1), 3% FCS, 200 mM, L-glutamine, 10 mg/ml Gentamicin, (100 nM) insulin, 0.2 nM T3, 1 μM dexamethasone, 0.25 mM IBMX, and 1 μM rosiglitazone can be used.

3 ml of this mixture are pipetted into each well. The gel is allowed to polymerize for 30 minutes in the 37° C. incubator. Once the gel is polymerized, 2 ml of adipogenesis differentiation medium is added on top of the gel only.

TABLE 3
Culture medium.

| | | |
|---|---|---|
| 3D Culture | 1 well | 6 wells |

| | | | | |
|---|---|---|---|---|
| Premix solution | 615 | μl | 4.1 | ml |
| Preadipocyte in adipogenesis | 327 | μl | 2.180 | ml |
| differentiation medium | | | | |
| (2 × 10⁵ cells per ml) | | | | |
| Bovine tail collagen | 2.0 | ml | 13.7 | ml |
| Total volume | 3 | ml | 20 | ml |

Change the medium every 2-3 d for the next 3 weeks.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of screening a test agent for adipogenic activity, said method comprising:
providing mammalian test cells with adipogenic potential wherein said cells are primed for, but withheld from, differentiation into adipocytes;
contacting said cells with said test agent; and
screening said test cells for an adipocyte phenotype wherein the presence of a feature characteristic of an adipocyte is an indicator that said test agent is adipogenic.

2. The method of claim 1, wherein said cells with adipogenic potential are cells selected from the group consisting of mesenchymal stem cells, papillary and reticular dermal fibroblasts, adipose derived stem/stromal cells, preadipocytes, myeloid precursors, myogenic precursors with adipogenic potential, vascular cells, embryonic ectoderm, and embryonic mesoderm.

3. The method of claim 2, wherein said cells with adipogenic potential are preadipocytes derived from skin, preadipocytes derived from liposuction, hair follicles, and preadipocytes derived from liposarcoma.

4. The method of claim 2, wherein said cells with adipogenic potential are selected from the group consisting of subcutaneous preadipocytes, and visceral preadipocytes.

5-8. (canceled)

9. The method of claim 2, wherein said cells are cells selected from the group consisting of 3T3-L1 cells, 3T3-F422A cells, 1246 cells, Ob1771 cells, TA1 cells, and 30A5 cells.

10. (canceled)

11. The method of claim 1, wherein said providing comprises contacting the cells with an adipocyte differentiation mix lacking at least one factor required for differentiation into an adipocyte.

12. The method of claim 11, wherein said adipocyte differentiation mix comprises one or more factors selected from the group consisting of IBMX, leptin, adponectin, glucose, adipogenic cytokine, adipogenic botanicals, dexamethasone, IGF-1, and insulin.

13. The method of claim 11, wherein said adipocyte differentiation mix comprises one or more factors selected from the group consisting of IBMX, dexamethasone, IGF-1, and insulin.

14. The method of claim 11, wherein said adipocyte differentiation mix does not one or more agents selected from the group consisting of insulin, IGF-1, antivirals, adipogenic cytokines, adipogenic factors, and adipogenic botanicals.

15. The method of claim 11, wherein said adipocyte differentiation mix does not include insulin and/or IGF-1.

16. The method of claim 11, wherein said adipocyte differentiation mix does not include an antiviral agent.

17. The method of claim 11, wherein said screening comprises detecting or quantifying a protein that is expressed specifically by adipocytes.

18. The method of claim 17, wherein said protein is selected from the group consisting of adiponectin, a lipid binding protein, and a transcription factor that promotes adipogenic transcriptomes.

19. The method of claim 1, wherein said screening comprises detecting or quantifying lipid accumulation in said cells wherein accumulation of lipid indicates that the cell has acquired characteristics of an adipocyte.

20. (canceled)

21. The method of claim 1, wherein said screening comprises comparing the results produced by said test agent on said cells with a positive control comprising the same cell type contacted with a complete adipocyte differentiation mix, wherein the absence of a significant difference between the test cells and the positive control is an indicator that the test agent is adipogenic.

22. The method of claim 21, wherein said complete adipocyte differentiation mix comprises IBMX, dexamethasone, and insulin.

23. The method of claim 1, wherein said screening comprises comparing the results produce by said test agent on said cells with a negative control comprising the same cell type not exposed to a differentiation mix wherein the absence of a significant difference between the test cells and the negative control is an indicator that the test agent is not adipogenic.

24. The method of claim 1, wherein said test cells are disposed in a plurality of different vessels or wells in a multi-well or multi-vessel device.

25.-30. (canceled)

31. The method of claim 1, wherein said cells are cultured in a 2-D cell culture.

32. The method of claim 1, wherein said cells are cultured in a 3-D cell culture.

33. (canceled)

34. The method of claim 1, wherein:
said test cells include subcutaneous preadipocytes and visceral preadipocytes; and
said screening comprises scoring as positive a test agent that induces adipogenesis in subcutaneous preadipocytes and that induces adipogenesis at a lesser amount or does not induce adipogenesis in visceral preadipocytes.

35. The method of claim 1, further comprising:
contacting fibroblasts with said test agent; and
screening said fibroblasts for changes in myofibroblast activity, wherein a test agent that shows adipogenic activity and inhibition of myofibroblast activity is a candidate agent for treatment or prophylaxis of cellulite.

36. (canceled)

37. The method of claim 1, wherein a test agent that screens positive is further validated by subcutaneous injection in a non-human mammal.

38. A cell culture system for screening a test agent for adipogenic activity, said cell culture system comprising:
one or more cell culture vessels containing mammalian cells having adipogenic potential wherein said cells are primed for, but withheld from differentiation into adipocytes.

39.-72. (canceled)
